(19) United States
(12) Patent Application Publication
(10) Pub. No.: US 2017/0239331 A1
(43) Pub. Date: Aug. 24, 2017

(54) FORMULATIONS FOR HISTATIN PROTECTIVES AND THERAPEUTICS

(71) Applicant: RAPID PATHOGEN SCREENING, INC., Sarasota, FL (US)

(72) Inventors: Robert P. Sambursky, Bradenton, FL (US); Robert W. Vandine, Montoursville, PA (US); Uma Mahesh Babu, Bradenton, FL (US)

(21) Appl. No.: 15/519,228

(22) PCT Filed: Oct. 8, 2015
(86) PCT No.: PCT/US15/54598

§ 371 Date: Apr. 14, 2017

Related U.S. Application Data
(60) Provisional application No. 62/064,164, filed on Oct. 15, 2014.
(60) Provisional application No. 62/064,137, filed on Oct. 15, 2014.
(60) Provisional application No. 62/064,151, filed on Oct. 15, 2014.
(60) Provisional application No. 62/065,911, filed on Oct. 20, 2014.
(60) Provisional application No. 62/065,920, filed on Oct. 20, 2014.
(60) Provisional application No. 62/065,935, filed on Oct. 20, 2014.

(51) Int. Cl. A61K 38/17; A61K 9/00

(52) U.S. Cl. CPC A61K 38/1709; A61K 9/0017

(57) ABSTRACT

Histatins may be used for epithelial wound prevention and healing in animals. Histatins may also be used for infection and disease prevention and healing in animals. For example, histatins may be included in gels, ointments, creams, tissue glues, patches, aerosol sprays, subcutaneous injections, subcutaneous infusions, intradermal injections, intradermal infusions, or any combination of these formulations. In some embodiments, peptide fragments from at least two different histatins are used.

### REFERENCE TO RELATED APPLICATIONS

This application claims one or more inventions which were disclosed in Provisional Application No. 62/064,164, filed Oct. 15, 2014, entitled “FORMULATIONS FOR HISTATIN PROTECTIVES AND THERAPEUTICS”, Provisional Application No. 62/064,137, filed Oct. 15, 2014, entitled “FORMULATIONS FOR HISTATIN THERAPEUTICS”, Provisional Application No. 62/064,151, filed Oct. 15, 2014, entitled “FORMULATIONS FOR HISTATIN THERAPEUTICS”, Provisional Application No. 62/065,911, filed Oct. 20, 2014, entitled “FORMULATIONS FOR HISTATIN THERAPEUTICS” and Provisional Application 62/065,920, filed Oct. 20, 2014, entitled “FORMULATIONS FOR HISTATIN THERAPEUTICS” and Provisional Application No. 62/065,935, filed Oct. 20, 2014, entitled “FORMULATIONS FOR HISTATIN PROTECTIVES AND THERAPEUTICS”. The benefit under 35 USC §119(e) of the United States provisional applications is hereby claimed, and the aforementioned applications are hereby incorporated herein by reference.

### BACKGROUND OF THE INVENTION

Field of the Invention

The invention pertains to the fields of wound, infection, and disease prevention and treatment. More particularly, the invention pertains to epithelial wound prevention and healing in animals, infection prevention and healing in animals, treating epithelial wounds in animals, and treating infections in animals using histatins.

Description of Related Art

Histatins have been shown in in vitro studies to be wound healing agents from saliva. More specifically, WO 2009/087117 (and its US equivalent, U.S. Patent Publication 2011/0178010), herein incorporated by reference, identified peptides of histatin, which had wound healing properties in vitro.

Histatin 1 (Hst-1) and Histatin 2 (Hst-2) have been identified as major wound-closing factors in human saliva (“Discovery of the Wound Healing Capacity of Salivary Histatins”, thesis of Menno Johannes Oudhoff, Academic Centre for Dentistry Amsterdam (ACTA), VU University Amsterdam and University of Amsterdam, The Netherlands, 2010, herein incorporated by reference). These studies were all done in vitro and cannot be translated to a finding for therapeutic or clinical use, especially since wound and disease healing are complex processes that need to be highly regulated in order to function properly.

Bovine mastitis is the inflammation of the mammary gland and udder tissue of cows. Bovine mastitis results when white blood cells are released into the mammary gland, usually in response to a bacterial infection. Escherichia coli and Streptococcus uberis are the most common bacteria that cause bovine mastitis, although many other types of bacteria have also been known to cause bovine mastitis. Bovine mastitis is the most common disease in U.S. dairy cattle and can be fatal. Certain forms of bovine mastitis may be spread from cow to cow through secondary contact with milking equipment.

Residual calf suckling has been shown to reduce the risk of mastitis in dual-purpose cows (González-Sedano et al., “Effect of Residual Calf Suckling on Clinical and Sub-Clinical Infections of Mastitis in Dual-Purpose Cows: Epidemiological Measurements”, Research in Veterinary Science, Vol. 89, pp. 362-366, 2010, herein incorporated by reference). Specifically, González-Sedano et al. reported that cows that did not suckle their calves after milking were an average of 6.59 times more likely to develop clinical mastitis than cows that did suckle after milking.

### SUMMARY OF THE INVENTION

Histatins may be used for epithelial wound prevention and healing in animals. Histatins may also be used for infection and disease prevention and healing in animals. For example, histatins may be included in gels, ointments, creams, tissue glues, patches, aerosol sprays, subcutaneous injections, subcutaneous infusions, intradermal injections, intradermal infusions, or any combination of these formulations. In some embodiments, peptide fragments from at least two different histatins are used.

In one preferred embodiment, a method of preventing epithelial wounds includes the step of administering a protective amount of a peptide or a peptide fragment of at least two histatins at an epithelial site. In a preferred embodiment, the protective amount of histatins reduces the likelihood of development of an epithelial wound at the epithelial site compared to unprotected epithelial sites.

In another preferred embodiment, a method of treating epithelial wounds includes the step of administering a therapeutic amount of a peptide or a peptide fragment of at least two histatins at a site of an epithelial wound. In a preferred embodiment, the therapeutic amount of histatins accelerates wound healing compared to untreated epithelial wounds.

In another preferred embodiment, a method of preventing infections includes the step of administering a protective amount of a peptide or a peptide fragment of at least two histatins at a vulnerable site prone to infection. In a preferred embodiment, the protective amount of histatins reduces the likelihood of development of an infection at the site compared to unprotected sites.

In another preferred embodiment, a method of treating infections includes the step of administering a therapeutic amount of a peptide or a peptide fragment of at least two histatins at a site of an infection. In a preferred embodiment, the therapeutic amount of histatin accelerates infection healing compared to untreated infected sites.

The histatins are preferably administered using gels, ointments, creams, tissue glues, patches, aerosol sprays, subcutaneous injections, subcutaneous infusions, intradermal injections, intradermal infusions, or any combination of these formulations.

The peptides or peptide fragments of the histatins preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

In some preferred embodiments, the histatins include a) at least a peptide fragment of histatin 5 and b) at least a peptide fragment of histatin 1, at least a peptide fragment of histatin 2 or a combination of at least a peptide fragment of histatin 1 and at least a peptide fragment of histatin 2.

In other preferred embodiments, the histatins include a) histatin 5 (SEQ ID NO: 30) and b) histatin 1 (SEQ ID NO: 4), histatin 2 (SEQ ID NO: 5), or a combination of histatin 1 and histatin 2.

In other embodiments, the histatins comprise the amino acid sequence SEQ ID NO: 33 in a cyclized form. In yet other embodiments, the histatins include a combination of peptide fragments of histatin 1, 2, and/or 5, and full length histatin 1, 2, and/or 5. In other preferred embodiments, the histatins include histatin 5 (SEQ ID NO: 30), either in a cyclized or noncyclized form, and cyclized histatin 1 (SEQ ID NO: 33).

In some embodiments, the infection to be prevented or treated is bovine mastitis. The histatin formulation is preferably administered by application to the teats of a milking cow.

In some embodiments, the wound to be treated or prevented in an animal is an incision, a laceration, an abrasion, a contusion, or a puncture.

In some embodiments, the histatins includes at least a peptide fragment of histatin 1 and at least a peptide fragment of histatin 5. In some embodiments, the histatins include histatin 1 (SEQ ID NO: 4) and histatin 5 (SEQ ID NO: 30). In other embodiments, the histatins include histatin 1 (SEQ ID NO: 4) and at least a peptide fragment of histatin 5. In other embodiments, the histatins include histatin 5 (SEQ ID NO: 30) and at least a peptide fragment of histatin 1. In some embodiments, at least one of the histatins is cyclized. In some embodiments, the cyclized histatin is cyclized histatin 1 (SEQ ID NO: 33).

### DETAILED DESCRIPTION OF THE INVENTION

Histatins are a family of naturally-occurring oral peptides first identified in human saliva that demonstrate direct anti-infective activity, potent anti-inflammatory properties, and stimulate epithelial wound healing in several tissue and organ culture systems. A research facility has developed a technique to isolate this natural substance, making it a potential topical treatment for wounds. Similar histatins have subsequently been identified in other higher mammals, including, but not limited to some non-human primates, dogs, cats, cows, pigs, and horses.

A “wound”, as defined herein, is an injury to living tissue, and can be caused by a cut, blow, or other impact. In most wounds, the skin or another external surface is cut or broken. Healing of those wounds using histatins 1 and 2 occurs by cell migration (epithelial migration, closing of an epithelial defect) and/or tissue regeneration. The regeneration occurs without induction of mitosis. In some embodiments, a wound is more specifically a physical manifestation of a breakdown of the protective function of the skin or other outer part of the body that normally provides a protective function, such as, for example, the cornea. In some embodiments, the wound reflects a loss of continuity of the epithelium. A cause of a wound may include, but is not limited to, surgery, a blow, a cut, contact with one or more chemicals, heat, cold, friction, a shear force, pressure, an ulcer, or a carcinoma.

An “infection”, as defined herein, is an invasion of the tissue of a host organism by a disease-causing agent and the reaction of the host to the agent and any toxins produced by the agent. In some embodiments, the invasion is by way of the skin tissue.

An “infectious disease” or “disease”, as defined herein, is an abnormal condition that affects an organism as a result of an infection.

In some embodiments, the histatins and the histatin sequences described herein are used as antimicrobial agents to prevent or treat infections in animals. In some embodiments, the histatins and the histatin sequences described herein are used to prevent or treat bovine mastitis.

U.S. Patent Publications 2013/0310326, and 2013/0310327, both published on Nov. 21, 2013, and entitled “HISTATIN FOR CORNEAL WOUND HEALING AND OCULAR SURFACE DISEASE”, and herein incorporated by reference, disclose histatins that may be used for corneal wound healing and as a treatment for ocular surface disease in humans and other animals.

The histatins and the histatin sequences described herein may be used as anti-fungal agents, antiviral agents, and/or antiparasitic agents for the skin and, preferably more specifically, the epithelium. In one embodiment, the histatins may be used to treat acanthamoeba epithelial infections. Acanthamoeba are one of the most common protozoa in soil and can also be found in fresh water or other habitats.

The antiviral efficacy of a histatin may be verified through the following type of in vitro test: 150 μL of a solution containing one or more histatins and a control solution are aliquoted into separate sterile screw cap microfuge tubes. 150 μL of stock virus in phosphate-buffered saline (PBS) are added to each tube containing the compounds and are mixed. The drug containing and control tubes are then incubated at 37° C. At 60 minutes of incubation, 300 μL of fresh tissue culture medium containing 20% fetal bovine serum is added to the tubes. Standard viral plaque assays are immediately performed to determine the residual viral titers present in each sample. Viral titers (PFU+1) are Log₁₀ converted and Log₁₀ reductions in titers from the control are calculated for each trial. The mean±SD Log reduction in titer for each virus are calculated for the two trials. Mean reductions in titer of at least one Log₁₀ are considered effective reductions. Mean reductions in titer of three Log₁₀ (99.9%) are considered virucidal reductions.

The antiparasitic efficacy of a histatin may be verified through the following type of in vitro test: 0.1 mL of Acanthamoeba inoculum is pipetted into 0.5 mL each of polyhexamethylene biguanide (PHMB) 0.02%, saline and two different concentrations of at least one histatin. The inoculated histatin and control samples are incubated at 30° C. for 24 hours. At 24 hours, 0.05 mL of the inoculated samples is removed from the mixtures and plated on non-nutrient agar overlaid with Enterobacter aerogenes using a glass rod to disperse the samples. This prevents a concentrated amount of histatin to inhibit bacterial growth. The overlay is prepared by spreading of 0.3 mL of the Enterobacter aerogenes slurry on a non-nutrient agar with a soft-tipped applicator. The plates are incubated at 30° C. in an air incubator. After another 24 hours, a second overlay of Enterobacter aerogenes is administered to assure the food source is available to the acanthamoeba without any effect of residual drug. All plates are monitored for the robust growth of acanthamoeba resulting in a mixture of sparse trophozoites and predominant cysts at days 7 and 14. Robust growth at day 7 terminates testing with a positive result. At day 14, all remaining plates (plates observed for the lack of robust growth) are vigorously sub-cultured with a soft-tipped applicator onto fresh non-nutrient agar overlaid with Enterobacter aerogenes. After 7 days incubation, all plates are monitored for the robust growth of acanthamoeba resulting in a mixture of sparse trophozoites and predominant cysts. For each drug concentration, positive growth is graded as a “1”, and no growth is denoted as “0”. Any incidence of positive growth after 24 hours is considered as survival. Negative growth at 24 hours is denoted as “kill”.

In a preferred embodiment, a peptide including at least one amino acid sequence of at least eight amino acids adjacently present in Histatin 1, 2, 3, and/or 5 is used to treat or prevent an epithelial wound. In other preferred embodiments, multiple histatin peptides or peptide fragments are used.

In one preferred embodiment, a method of preventing epithelial wounds includes the step of administering a protective amount of at least a portion of a histatin peptide at a vulnerable epithelial site prone to an epithelial wound. In another preferred embodiment, a method of preventing epithelial wounds includes the step of administering a protective amount of at least a portion of at least two histatin peptides at a vulnerable epithelial site prone to an epithelial wound.

In another preferred embodiment, a method of treating epithelial wounds includes the step of administering a therapeutic amount of at least a portion of a histatin peptide at a site of an epithelial wound. In another preferred embodiment, a method of treating epithelial wounds includes the step of administering a therapeutic amount of at least a portion of at least two histatin peptides at a site of an epithelial wound.

In one preferred embodiment, a method of preventing infections includes the step of administering a protective amount of at least a portion of a histatin peptide at a vulnerable site prone to an infection. In another preferred embodiment, a method of preventing infections includes the step of administering a protective amount of at least a portion of at least two histatin peptides at a vulnerable site prone to an infection.

In another preferred embodiment, a method of treating infections includes the step of administering a therapeutic amount of at least a portion of a histatin peptide at a site of an infection. In another preferred embodiment, a method of treating infections includes the step of administering a therapeutic amount of at least a portion of at least two histatin peptides at a site of an infection.

The histatins are preferably administered using gels, ointments, creams, tissue glues, patches, aerosol sprays, subcutaneous injections, subcutaneous infusions, intradermal injections, intradermal infusions, or any combination of these formulations. In one preferred embodiment, a protective amount of histatin reduces the likelihood of development of an infection at a vulnerable site prone to infection compared to sites not protected with histatin. In another preferred embodiment, the protective amount of histatin reduces the likelihood of development of an epithelial wound at an epithelial site compared to epithelial sites not protected with histatin. In one preferred embodiment, a therapeutic amount of histatin accelerates infection healing compared to infections not treated with histatin. In another preferred embodiment, the therapeutic amount of histatin accelerates wound healing compared to epithelial wounds not treated with histatin.

In some preferred embodiments, the histatin concentration is between approximately 0.1 μg/mL and approximately 1000 mg/mL. In other preferred embodiments, the histatin concentration is between approximately 0.1 μg/mL and 100 μg/mL. In still other preferred embodiments, the histatin concentration is between approximately 0.1 μg/mL and 10 μg/mL. In some preferred embodiments, the histatin concentration is greater than or equal to approximately 1 μM.

The administering step may be repeated multiple times per day and/or for a plurality of days. In one preferred embodiment, this step is repeated at least one time a day for a plurality of days. In another preferred embodiment, the step is repeated chronically at least one time a day. In some preferred embodiments, the step is repeated up to hourly for a plurality of days. In another preferred embodiment, the step is repeated at least two times a day for a plurality of days. In yet another preferred embodiment, the step is repeated at least three times a day for a plurality of days, for example for seven days. In another preferred embodiment, the step is repeated four times a day for five days.

In one preferred embodiment, at least one of the histatins is a peptide including 8 to 44 amino acids. In some preferred embodiments, at least one of the peptides is an L-peptide. In other preferred embodiments, at least one of the peptides is a cyclic peptide.

In some preferred embodiments, the amino acid sequence of the histatin peptide is one or more of SEQ ID NOS: 1 through 33, or any combinations of these sequences. In alternative embodiments, one or more of the amino acid sequences have a substitution, deletion and/or insertion of up to 3 amino acids. In other alternative embodiments, one or more of the amino acid sequences have a substitution, a deletion and/or an insertion of two or less amino acids. In other alternative embodiments, one or more of the amino acid sequences have a substitution, a deletion, and/or an insertion in one amino acid.

The SEQ ID NO: 4 peptide is also known as Histatin 1 (Hst-1). Note that the first serine in this amino acid sequence may be a phosphoserine. The SEQ ID NO: 5 peptide is also known as Histatin 2 (Hst-2, also equivalent to amino acids 12-38 of Hst-1). The SEQ ID NO: 6 peptide is also known as Histatin 3 (Hst-3). The SEQ ID NO: 30 peptide is also known as Histatin 5 (Hst-5). Parts and fragments of each of these amino acid sequences may be used, alone or in combination, including but not limited to SEQ ID NOS: 1-3, 7-29 (for Histatin 1, Histatin 2 and Histatin 3) and SEQ ID NO: 32 (for Histatin 5) to facilitate wound closure in the embodiments described herein. While the L stereoisomer of the amino acids is preferred for the amino acid sequences described herein, D stereoisomers may alternatively be used. Alternatively, amino acid sequences that include these histatins and other amino acids, for example SEQ ID NO: 33, which is a sortase cyclized histatin (including all of Histatin 1), may be used in the embodiments described herein. Any histatin sequences could be cyclized and used in the embodiments described herein.

In one preferred embodiment, a method of preventing epithelial wounds includes the step of administering a protective amount of at least a peptide fragment of at least two histatins at a vulnerable epithelial site prone to epithelial wounds. The histatins are preferably administered using gels, ointments, creams, tissue glues, patches, aerosol sprays, subcutaneous injections, subcutaneous infusions, intradermal injections, intradermal infusions, or any combination of these formulations.

In another preferred embodiment, a method of treating epithelial wounds includes the step of administering a therapeutic amount of at least a peptide fragment of at least two histatins at a site of an epithelial wound. The histatins are preferably administered using gels, ointments, creams, tissue glues, patches, aerosol sprays, subcutaneous injections, subcutaneous infusions, intradermal injections, intradermal infusions, or any combination of these formulations.

In one preferred embodiment, a method of preventing infections includes the step of administering a protective amount of at least a peptide fragment of at least two histatins at a vulnerable site prone to infections. The histatins are preferably administered using gels, ointments, creams, tissue glues, patches, aerosol sprays, subcutaneous injections, subcutaneous infusions, intradermal injections, intradermal infusions, or any combination of these formulations.

In another preferred embodiment, a method of treating infections includes the step of administering a therapeutic amount of at least a peptide fragment of at least two histatins at a site of an infection. The histatins are preferably administered using gels, ointments, creams, tissue glues, patches, aerosol sprays, subcutaneous injections, subcutaneous infusions, intradermal injections, intradermal infusions, or any combination of these formulations.

In preferred embodiments, using a histatin amino acid sequence that promotes skin wound closure (for example, a histatin amino acid sequence present in histatin 1 or histatin 2) in combination with a histatin amino acid sequence with antimicrobial properties (for example, a histatin amino acid sequence present in histatin 5), has synergistic protection and healing effects. This strategy combines the direct effects that histatin 1 and histatin 2 have on wound closure with the indirect effects the antimicrobial properties of histatin 5 have on wound closure. More specifically, histatins 1 and 2 promote wound closure, while histatin 5 prevents microbial infection, thereby creating a better environment for wound healing and infection fighting.

In a preferred embodiment, the protective amount of histatin prevents or reduces the risk of an epithelial wound at an epithelial site compared to epithelial sites not treated with histatin. In another preferred embodiment, the protective amount of histatin prevents or reduces the risk of an infection at a treated site compared to sites not treated with histatin. In another preferred embodiment, the therapeutic amount of histatin accelerates wound healing compared to epithelial wounds not treated with histatin. In another preferred embodiment, the therapeutic amount of histatin accelerates infection healing compared to infections not treated with histatin. The peptide fragments of the histatins preferably includes at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

Some preferred embodiments use amino acid sequences from Hst-1 and/or Hst-2 in combination with amino acid sequences from Hst-5 to protect epithelial sites, to treat epithelial wounds, to prevent infections, or to treat infections. In these embodiments, one or more amino acid sequences from Hst-1 and/or Hst-2 are chosen, and one or more amino acid sequences from Hst-5 are chosen. In some embodiments, the full length Histatin 1 (SEQ ID NO: 4), full length Histatin 2 (SEQ ID NO: 5), and/or the full length Histatin 5 (SEQ ID NO: 30) are used. In other embodiments, portions of Hst-1, Hst-2, and/or Hst-5 are used. For example, SEQ ID NO: 29, which is equivalent to amino acids 20-32 of Histatin 1, may be a preferred amino acid sequence to use for wound closure in some embodiments. In other examples, peptides including SEQ ID NO: 32, a peptide fragment of Histatin 1 and Histatin 2 that appears to be a core motif for wound closure, may be used. Other preferred sequences from Hst-1 and Hst-2 include, but are not limited to, SEQ ID NO: 8, SEQ ID NO: 9 and SEQ ID NO: 13. As another example, SEQ ID NO: 31, a fragment of Histatin 5 (Gusman et al., “Salivary Histatin 5 is an inhibitor of Both Host and Bacterial Enzymes Implicated in Periodontal Disease”, Infect. Immun. 2001, 69(3): 1402, pp. 1402-1408, herein incorporated by reference), may be used, preferably in combination with Histatin 1 or Histatin 2 or fragments thereof. In other preferred embodiments, fragments of Hst-1 or Hst-2 are used with full length Hst-5 (SEQ ID NO: 30) or full length Hst-1 (SEQ ID NO: 4) or Hst-2 (SEQ ID NO: 5) are used with fragments of Hst-5 (for example, SEQ ID NO: 31). In yet other embodiments, any combination of fragments of Hst-1 and/or Hst-2, full length Hst-1 and/or Hst-2, fragments of Hst-5, or full length Hst-5 may be used. In some preferred embodiments, the concentration of the Hst-5 peptide used is greater than or equal to approximately 1 μM.

The amino acids and the peptides described herein may include at least one functional grouping (for example, an amine and/or carboxylic group) protected with a protective grouping in some embodiments. Since the peptides are applied to tissue, skin, or a wound, a protected form of the peptide may be preferred to resist degradation. The form of protection needs to be biologically compatible and compatible with pharmaceutical use. Some examples include, but are not limited to, the acylation or the acetylation of the amino-terminal ends, cyclization or the amidation or the esterfication of the carboxy-terminal ends. Thus, the peptides described herein may be used in a protected form.

The peptides described herein may be made by traditional chemical synthesis, enzymatic synthesis, or any other method known in the art.

The peptides preferably include at least 8 amino acids. In one preferred embodiment, the peptides include a range of 8 to 44 amino acids, but the peptides may alternatively include more than 44 amino acids.

Histatins and peptide portions or peptide fragments of histatins may be used to prevent epithelial wound formation, prevent infections, accelerate epithelial wound healing, or accelerate infection healing in animals. In preferred embodiments, histatin 1 (Hst-1), histatin 2 (Hst-2), histatin 5 (Hst-5), peptide fragments of Hst-1, Hst-2, or Hst-5, or any combinations thereof may be used. In other embodiments, histatin 3 (Hst-3) or the D-enantiomer of histatin 2 (D-Hst-2), or peptide fragments thereof, may be used. Any combinations of any of the histatins may be used. In preferred embodiments, histatin concentrations between 0.1 μg/mL and 1000 mg/mL may be used. Peptides with amino acid SEQ ID NOS: 1-33, histatins known in the art, the peptides disclosed in WO 2009/087117 or the peptides disclosed in Dr. Menno Johannes Oudhoff's thesis, “Discovery of the Wound-Healing Capacity of Salivary Histatins”, 2010, department of Oral Biochemistry of the Academic Centre for Dentistry Amsterdam (ACTA), VU University Amsterdam and University of Amsterdam, The Netherlands, herein incorporated by reference, may be used.

In one preferred embodiment, histatin 1 (Hst-1) or histatin 2 (Hst-2) in combination with histatin 5 (Hst-5), peptide fragments of Hst-1 or Hst-2 in combination with peptide fragments of Hst-5, or any combination, are used. Hst-5 inhibits production of Matrix Metalloproteases (MMPs).

The combination of the Hst-1/Hst-2 healing properties with the Hst-5 inhibiting MMPs should be very effective. In some preferred embodiments, a concentration of at least approximately 1 μM of Hst-5, or a fragment of Hst-5, is used.

In one preferred embodiment, a cyclic version of SEQ ID NO: 33 is used in combination with SEQ ID NO: 30, either in a cyclized or non-cyclized form.

Histatins may be administered to animals with an epithelial wound or to an epithelial site to prevent an epithelial wound or to animals with an infection or to a site to prevent an infection. Some methods of administration include, but are not limited to, incorporating the histatin into gels, ointments, creams, tissue glues (to transiently seal epithelial injuries), patches, aerosol sprays, subcutaneous injections, subcutaneous infusions, intradermal injections, intradermal infusions, or combinations of these formulations.

The histatins may be administered in any combination of daily treatments for any number of days in order to produce protective or therapeutic results. In one preferred embodiment, the histatin is administered at least once a day for a plurality of days. In another preferred embodiment, the histatin is administered at least once a day chronically (for an extended period of time). In another preferred embodiment, the step may be repeated two, three, four, five times or more, or hourly, for a plurality of days or chronically. In one example, the histatin is repeated three times a day for seven days. In another example, histatin is administered four times a day for five days.

Histatin Formulations for Epithelial Wound Prevention, Epithelial Wounds, Infection Prevention, and Infections

In some embodiments, a formulation for histatin for protection of epithelial sites includes a total of approximately 25-150 μg/mL of a combination of a histatin 1 fragment and a histatin 5 fragment in an applying vehicle. In some preferred embodiments, a formulation for histatin for protection of epithelial sites includes a total of approximately 50-100 μg/mL of a combination of a histatin 1 fragment and a histatin 5 fragment in an applying vehicle. The fragment may include the entire histatin 1 or histatin 5 sequence, or just a portion of one or both of those sequences. The weight-to-weight ratio of histatin 1 to histatin 5 is preferably 1:1, 2:1, 3:1, 4:1, 5:1, 1:2, 1:3, 1:4, 1:5, or within a range inclusive of any two of these ratios. The amount of each histatin in the formulation is more preferably in the range of 50-75 wt % of the histatin 1 fragment (25 to 75 μg/mL) and 25-50 wt % of the histatin 5 fragment (12.5 to 50 μg/mL) with respect to the total weight of histatins in the formulation. In some preferred embodiments of this formulation, both the histatin 1 and the histatin 5 fragment are cyclized. In other preferred embodiments, one of the fragments is cyclized. In other embodiments, neither of the fragments is cyclized.

In some embodiments, a formulation for histatin for treating epithelial wounds includes a total of approximately 25-150 μg/mL of a combination of a histatin 1 fragment and a histatin 5 fragment in an applying vehicle. In some preferred embodiments, a formulation for histatin for treating epithelial wounds includes a total of approximately 50-100 μg/mL of a combination of a histatin 1 fragment and a histatin 5 fragment in an applying vehicle. The fragment may include the entire histatin 1 or histatin 5 sequence, or just a portion of one or both of those sequences. The weight-to-weight ratio of histatin 1 to histatin 5 is preferably 1:1, 2:1, 3:1, 4:1, 5:1, 1:2, 1:3, 1:4, 1:5, or within a range inclusive of any two of these ratios. The amount of each histatin in the formulation is more preferably in the range of 50-75 wt % of the histatin 1 fragment (25 to 75 μg/mL) and 25-50 wt % of the histatin 5 fragment (12.5 to 50 μg/mL) with respect to the total weight of histatins in the formulation. In some preferred embodiments of this formulation, both the histatin 1 and the histatin 5 fragment are cyclized. In other preferred embodiments, one of the fragments is cyclized. In other embodiments, neither of the fragments is cyclized.

In some embodiments, a formulation for histatin for protection of vulnerable sites prone to infection includes a total of approximately 25-150 μg/mL of a combination of a histatin 1 fragment and a histatin 5 fragment in an applying vehicle. In some preferred embodiments, a formulation for histatin for protection of vulnerable sites prone to infection includes a total of approximately 50-100 μg/mL of a combination of a histatin 1 fragment and a histatin 5 fragment in an applying vehicle. The fragment may include the entire histatin 1 or histatin 5 sequence, or just a portion of one or both of those sequences. The weight-to-weight ratio of histatin 1 to histatin 5 is preferably 1:1, 2:1, 3:1, 4:1, 5:1, 1:2, 1:3, 1:4, 1:5, or within a range inclusive of any two of these ratios. The amount of each histatin in the formulation is more preferably in the range of 50-75 wt % of the histatin 1 fragment (25 to 75 μg/mL) and 25-50 wt % of the histatin 5 fragment (12.5 to 50 μg/mL) with respect to the total weight of histatins in the formulation. In some preferred embodiments of this formulation, both the histatin 1 and the histatin 5 fragment are cyclized. In other preferred embodiments, one of the fragments is cyclized. In other embodiments, neither of the fragments is cyclized.

In some embodiments, a formulation for histatin for treating infections includes a total of approximately 25-150 μg/mL of a combination of a histatin 1 fragment and a histatin 5 fragment in an applying vehicle. In some preferred embodiments, a formulation for histatin for treating infections includes a total of approximately 50-100 μg/mL of a combination of a histatin 1 fragment and a histatin 5 fragment in an applying vehicle. The fragment may include the entire histatin 1 or histatin 5 sequence, or just a portion of one or both of those sequences. The weight-to-weight ratio of histatin 1 to histatin 5 is preferably 1:1, 2:1, 3:1, 4:1, 5:1, 1:2, 1:3, 1:4, 1:5, or within a range inclusive of any two of these ratios. The amount of each histatin in the formulation is more preferably in the range of 50-75 wt % of the histatin 1 fragment (25 to 75 μg/mL) and 25-50 wt % of the histatin 5 fragment (12.5 to 50 μg/mL) with respect to the total weight of histatins in the formulation. In some preferred embodiments of this formulation, both the histatin 1 and the histatin 5 fragment are cyclized. In other preferred embodiments, one of the fragments is cyclized. In other embodiments, neither of the fragments is cyclized.

In one preferred embodiment, the histatin 1 fragment is a cyclized version of SEQ ID NO: 33. In another preferred embodiment, the histatin 5 fragment is SEQ ID NO: 30. In another preferred embodiment, the histatin 5 fragment is a cyclized version of SEQ ID NO: 30. In another preferred embodiment, the histatin 1 fragment is a cyclized version of SEQ ID NO: 33 and the histatin 5 fragment is SEQ ID NO: 30 (either in a cyclic or linear form).

In other preferred embodiments, the histatin formulation comprises only Histatin 1. In some of these embodiments, the histatin 1 fragment is a cyclized version of SEQ ID NO: 33.

In some embodiments, the preferred vehicle in histatin formulations is a mixture of 50 wt %-65 wt % white petrolatum and 35 wt %-50 wt % purified water with 2 wt %-2.5 wt %, preferably 2.3 wt %, of a modified carboxymethycellulose polymer or liquid paraffin together with ethylene glycol, aloe, or propylene glycol.

Other alternative or additional ingredients include, but are not limited to, liquid paraffin, ethylene glycol, aloe barbadensis (aloe vera) gel, monostearate, stearic acid, paraffin wax, aluminum sulfate, calcium acetate, cetearyl alcohol, mineral oil, maltodextrin, white wax, and any combination of these ingredients.

Some preferred preservatives to be used in the formulation include, but are not limited to, potassium sorbate, propylparaben, Benzalkonium chloride (BAK), or any combination of these preservatives.

These formulations may be administered to animals with an epithelial wound, prone to an epithelial wound, with an infection, or prone to an infection. Some methods of administration include, but are not limited to, incorporating the histatin into gels, ointments, creams, tissue glues (to transiently seal epithelial injuries), patches, aerosol sprays, subcutaneous injections, subcutaneous infusions, intradermal injections, intradermal infusions, or any combination of these formulations. In one preferred embodiment, the histatin formulation is between an ointment and a cream in physical properties.

These formulations may be administered in any combination of daily treatments for any number of days in order to produce protective or therapeutic results. In one preferred embodiment, the histatin is administered at least once a day for a plurality of days. In another preferred embodiment, the histatin is administered at least once a day chronically (for an extended period of time). In another preferred embodiment, the step may be repeated two, three, four, five times or more, or hourly, for a plurality of days or chronically. In one example, the histatin is repeated three times a day for seven days. In another example, histatin is administered four times a day for five days.

In some embodiments, the histatin formulation is administered to prevent or reduce the risk of mastitis in a milking cow. The histatin formulation is preferably administered by application to the teats of the cow after the cow is released from the milking parlor, preferably within 5 minutes of completion of a milking event. The histatin formulation preferably includes histatin 1 and histatin 5 mixed in a teat guard vehicle. The teat guard vehicle preferably includes purified water or deionized water and at least one ingredient of dimethicones, stearic acid, glycerin, an emollient, an emulsifier, a thickening agent, and a stabilizer. The histatin formulation is preferably applied to the teat surface and over the sphincter and streak canal of the teat and quickly dries and forms a protective barrier. The protective barrier remains intact until the cow returns to the milking parlor for the next milking but substantially dissolves and is wiped off when the milker uses a wet cloth to wipe down the udder and teat prior to the milking, so that little, if any, of the histatin formulation ends up in the collected milk.

Deionized water is useful for skin care where the presence of impurities may be undesirable.

Dimethicones, also known as polydimethylsiloxanes, stay on or near the surface of the skin. Not only are the molecules too big to physically enter past the upper living cells (they associate with the upper layer of drying skin), but they also cannot penetrate cell membranes due to their large size. Dimethicones evaporate quickly after helping to carry oils into the top layer of epidermis. From there, the oils may be absorbed by the skin. Dimethicones form a protective layer which helps prevent transdermal water loss. Dimethicones act to help seal moisture into the outer layer of skin, which helps prevent many kinds of damage.

Stearic acid is a useful saturated fatty acid that comes from many vegetable fats and oils and is very stable in storage.

Glycerin, also known as glycerol and glycerine, is a humectant, meaning that it attracts moisture to your skin. Glycerin is a neutral, sweet-tasting, colorless, thick liquid that freezes to a gummy paste and has a high boiling point. Glycerin can be dissolved into water or alcohol, but not oils. On the other hand, many things will dissolve into glycerin easier than they do into water or alcohol. Glycerin is also highly hygroscopic, which means that it absorbs water from the air. For example, a bottle of pure glycerin exposed to humid air takes moisture from the air, and eventually the pure glycerin becomes 80 percent glycerin and 20 percent water.

In some embodiments, the emulsifier is cetyl alcohol. Cetyl alcohol is derived from naturally occurring fatty acids from coconut oil and is a secondary emulsifier that thickens or adds body to lotions. Cetyl alcohol and stearyl alcohol together create a cetearyl alcohol that forms an occlusive film to keep skin moisture from evaporating and gives skin a velvety feeling.

In some embodiments, the emollient is isopropyl myristate. Isopropyl myristate is used as an emollient and lubricant in pre-shaves, aftershaves, shampoos, bath oils, antiperspirants, deodorants, and various creams and lotions. Isopropyl myristate spreads very easily and promotes a dry feeling by reducing the greasy feel of skin products by replacing other, oilier ingredients.

In some embodiments, xanthan gum is included in a teat guard to serve as a thickening agent and a stabilizer. Xanthan gum is a natural polysaccharide used in dairy products and salad dressings. Xanthan gum prevents ice crystals from forming in ice creams. Xanthan gum is the stabilization and binding agent of cosmetic products.

All of the patent and nonpatent references discussed herein are incorporated herein by reference.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A method of reducing a risk of an infection at a vulnerable site of an animal, comprising the step of:
administering a protective amount of at least one medicament at the vulnerable site of the animal, the at least one medicament comprising at least a first peptide and a second peptide selected from the group consisting of:
the first peptide comprising a first histatin and the second peptide comprising a second histatin;
the first peptide comprising a fragment of the first histatin and the second peptide comprising a fragment of the second histatin;
the first peptide comprising the first histatin and the second peptide comprising a fragment of the second histatin; and
the first peptide comprising a fragment of the first histatin and the second peptide comprising the second histatin;
such that the at least one medicament reduces the risk of the infection at the vulnerable site relative to an untreated site.

2. The method of claim 1, comprising administering the medicament using gels, ointments, creams, patches, aerosol sprays, subcutaneous injections, subcutaneous infusions, intradermal injections, intradermal infusions, or any combination of gels, ointments, patches, creams, aerosol sprays, subcutaneous injections, subcutaneous infusions, intradermal injections, and intradermal infusions.

3. The method of claim 1, comprising administering the medicament using tissue glue.

4. The method of claim 1, wherein a histatin concentration of each histatin in the medicament is between approximately 0.1 μg/mL and approximately 100 μg/mL.

5. The method of claim 4, wherein a total concentration of the histatins in the medicament is between approximately 25 μg/mL and 150 μg/mL.

6. The method of claim 1, comprising repeating the step of administering a protective amount of at least one medicament at the vulnerable site of the animal at a schedule selected from the group consisting of:
a) at least one time a day for a plurality of days;
b) chronically at least one time a day;
c) up to hourly for a plurality of days;
d) at least two times a day for a plurality of days;
e) at least three times a day for a plurality of days;
f) three times a day for seven days; and
g) four times a day for five days.

7. The method of claim 1, wherein the medicament comprises at least two different amino acid sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

8-9. (canceled)

10. The method of claim 1, wherein at least one of the first peptide and the second peptide in the medicament is a cyclic peptide.

11. The method of claim 1, wherein the medicament comprises:
a) the first peptide selected from the group consisting of:
i) histatin 1;
ii) a fragment of histatin 1;
iii) histatin 2;
iv) a fragment of histatin 2;
v) histatin 1 and histatin 2;
vi) histatin 1 and a fragment of histatin 2;
vii) histatin 2 and a fragment of histatin 1; and
viii) a fragment of histatin 1 and a fragment of histatin 2; and
b) the second peptide selected from the group consisting of:
i) histatin 5; and
ii) a fragment of histatin 5.

12. The method of claim 1, wherein the second peptide comprises an amino acid sequence selected from the group consisting of SEQ ID NO: 30 and SEQ ID NO: 31.

13. The method of claim 1, wherein the first peptide comprises an amino acid sequence selected from the group consisting of SEQ ID NO: 4; SEQ ID NO: 5; SEQ ID NO: 8; SEQ ID NO: 9; SEQ ID NO: 13; SEQ ID NO: 29; and SEQ ID NO: 33.

14. The method of claim 11, wherein the first peptide is SEQ ID NO: 33 and the second peptide is SEQ ID NO: 30.

15. The method of claim 1, wherein the medicament comprises:
a) the first peptide selected from the group consisting of:
i) histatin 1;
ii) histatin 2; and
iii) a combination of histatin 1 and histatin 2; and
b) the second peptide comprising histatin 5.

16-17. (canceled)

18. The method of claim 1, wherein the vulnerable site of the animal comprises at least one teat of an udder of a milking cow.

19. The method of claim 18, wherein the infection is bovine mastitis.

20. The method of claim 18, comprising administering the medicament using a teat guard.

21. The method of claim 20, wherein the teat guard comprises water, at least one dimethicone, stearic acid, glycerin, at least one emollient, at least one emulsifier, and at least one thickening agent.

22. The method of claim 21, wherein the at least one emollient comprises isopropyl myristate, the at least one emulsifier comprises cetyl alcohol, and the at least one thickening agent comprises xanthan gum.

23. The method of claim 20, wherein the medicament is applied to the at least one teat after a first milking event.

24. The method of claim 23 further comprising the step of wiping the teat with a wet cloth prior to a second milking event after the first milking event to remove at least a portion of the medicament from the teat.

25. A method of treating an infection of an animal, comprising the step of:
administering a therapeutic amount of at least one medicament to the infection of the animal, the at least one medicament comprising a first peptide and a peptide selected from the group consisting of:
the first peptide comprising a first histatin and the second peptide comprising a second histatin;
the first peptide comprising the first histatin and the second peptide comprising a fragment of the second histatin;
the first peptide comprising a fragment of the first histatin and the second peptide comprising a fragment of the second histatin; and
the first peptide comprising a fragment of the first histatin and the second peptide comprising the second histatin.

26. The method of claim 25, comprising administering the medicament using gels, ointments, creams, patches, aerosol sprays, subcutaneous injections, subcutaneous infusions, intradermal injections, intradermal infusions, or any combination of gels, ointments, patches, creams, aerosol sprays, subcutaneous injections, subcutaneous infusions, intradermal injections, and intradermal infusions.

27. The method of claim 25, comprising administering the medicament using tissue glue.

28. The method of claim 25, wherein a histatin concentration of each histatin in the medicament is between approximately 0.1 μg/mL and approximately 100 μg/mL.

29. The method of claim 28, wherein a total concentration of the histatins in the medicament is between approximately 25 μg/mL and 150 μg/mL.

30. The method of claim 25, comprising repeating the step of administering a therapeutic amount of at least one medicament at the vulnerable site of the animal at a schedule selected from the group consisting of:
a) at least one time a day for a plurality of days;
b) chronically at least one time a day;
c) up to hourly for a plurality of days;
d) at least two times a day for a plurality of days;
e) at least three times a day for a plurality of days;
f) three times a day for seven days; and
g) four times a day for five days.

31. The method of claim 25, wherein the therapeutic amount of histatin in the medicament accelerates infection healing compared to infections not treated with the medicament.

32. The method of claim 25, wherein the medicament comprises at least two different amino acid sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

33-34. (canceled)

35. The method of claim 25, wherein at least one of the first peptide and the second peptide in the medicament is a cyclic peptide.

36. The method of claim 25, wherein the medicament comprises:
a) the first peptide selected from the group consisting of:
i) histatin 1;
ii) a fragment of histatin 1;
iii) histatin 2;
iv) a fragment of histatin 2;
v) histatin 1 and histatin 2;
vi) histatin 1 and a fragment of histatin 2;
vii) histatin 2 and a fragment of histatin 1; and
viii) a fragment of histatin 1 and a fragment of histatin 2; and
b) the second peptide selected from the group consisting of:
i) histatin 5; and
ii) a fragment of histatin 5.

37. The method of claim 25, wherein the second peptide comprises an amino acid sequence selected from the group consisting of SEQ ID NO: 30 and SEQ ID NO: 31.

38. The method of claim 25, wherein the first peptide comprises an amino acid sequence selected from the group consisting of SEQ ID NO: 4; SEQ ID NO: 5; SEQ ID NO: 8; SEQ ID NO: 9; SEQ ID NO: 13; SEQ ID NO: 29; and SEQ ID NO: 33.

39. The method of claim 36, wherein the first peptide is SEQ ID NO: 33 and the second peptide is SEQ ID NO: 30.

40. The method of claim 25, wherein the medicament comprises:
a) the first peptide selected from the group consisting of:
i) histatin 1;
ii) histatin 2; and
iii) a combination of histatin 1 and histatin 2; and
b) the second peptide comprising histatin 5.

41-42. (canceled)

43. The method of claim 25, wherein the vulnerable site of the animal comprises at least one teat of an udder of a milking cow.

44. The method of claim 43, wherein the infection is bovine mastitis.

45. The method of claim 43, comprising administering the medicament using a teat guard.

46. The method of claim 45, wherein the teat guard comprises water, at least one dimethicone, stearic acid, glycerin, at least one emollient, at least one emulsifier, and at least one thickening agent.

47. The method of claim 46, wherein the at least one emollient comprises isopropyl myristate, the at least one emulsifier comprises cetyl alcohol, and the at least one thickening agent comprises xanthan gum.

48. The method of claim 45, wherein the medicament is applied to the at least one teat after a first milking event.

49. The method of claim 48 further comprising the step of wiping the teat with a wet cloth prior to a second milking event after the first milking event to remove at least a portion of the medicament from the teat.

50-116. (canceled)


SEQUENCE LISTING (33 sequences)

SEQ ID NO: 1 - protein, 7 aa, Artificial sequence
  synthetic
SNYLYDN

SEQ ID NO: 2 - protein, 9 aa, Artificial sequence
  synthetic
REFPFYGDY

SEQ ID NO: 3 - protein, 6 aa, Artificial sequence
  synthetic
EKHHSH

SEQ ID NO: 4 - protein, 38 aa, Artificial sequence
  synthetic
DSHEKRHHGYRRKFHEKHHSHREFPFYGDYGSNYLYDN

SEQ ID NO: 5 - protein, 27 aa, Artificial sequence
  synthetic
RKFHEKHHSHREFPFYGDYGSNYLYDN

SEQ ID NO: 6 - protein, 32 aa, Artificial sequence
  synthetic
DSHAKRHHGYKRKFHEKHHSHRGYRSNYLYDN

SEQ ID NO: 7 - protein, 15 aa, Artificial sequence
  synthetic
EKHHSHREFPFYGDY

SEQ ID NO: 8 - protein, 17 aa, Artificial sequence
  synthetic
REFPFYGDYGSNYLYDN

SEQ ID NO: 9 - protein, 19 aa, Artificial sequence
  synthetic
SHREFPFYGDYGSNYLYDN

SEQ ID NO: 10 - protein, 21 aa, Artificial sequence
  synthetic
HHSHREFPFYGDYGSNYLYDN

SEQ ID NO: 11 - protein, 23 aa, Artificial sequence
  synthetic
EKHHSHREFPFYGDYGSNYLYDN

SEQ ID NO: 12 - protein, 25 aa, Artificial sequence
  synthetic
FHEKHHSHREFPFYGDYGSNYLYDN

SEQ ID NO: 13 - protein, 19 aa, Artificial sequence
  synthetic
RKFHEKHHSHREFPFYGDY

SEQ ID NO: 14 - protein, 15 aa, Artificial sequence
  synthetic
EKHHSHREFPFYGDY

SEQ ID NO: 15 - protein, 21 aa, Artificial sequence
  synthetic
RKFHEKHHSHREFPFYGDYGS

SEQ ID NO: 16 - protein, 17 aa, Artificial sequence
  synthetic
EKHHSHREFPFYGDYGS

SEQ ID NO: 17 - protein, 23 aa, Artificial sequence
  synthetic
RKFHEKHHSHREFPFYGDYGSNY

SEQ ID NO: 18 - protein, 19 aa, Artificial sequence
  synthetic
EKHHSHREFPFYGDYGSNY

SEQ ID NO: 19 - protein, 25 aa, Artificial sequence
  synthetic
RKFHEKHHSHREFPFYGDYGSNYLY

SEQ ID NO: 20 - protein, 21 aa, Artificial sequence
  synthetic
EKHHSHREFPFYGDYGSNYLY

SEQ ID NO: 21 - protein, 17 aa, Artificial sequence
  synthetic
RKFHEKHHSHREFPFYG

SEQ ID NO: 22 - protein, 13 aa, Artificial sequence
  synthetic
EKHHSHREFPFYG

SEQ ID NO: 23 - protein, 19 aa, Artificial sequence
  synthetic
HHSHREFPFYGDYGSNYLY

SEQ ID NO: 24 - protein, 17 aa, Artificial sequence
  synthetic
SHREFPFYGDYGSNYLY

SEQ ID NO: 25 - protein, 15 aa, Artificial sequence
  synthetic
SHREFPFYGDYGSNY

SEQ ID NO: 26 - protein, 15 aa, Artificial sequence
  synthetic
FPFYGDYGSNYLYDN

SEQ ID NO: 27 - protein, 13 aa, Artificial sequence
  synthetic
FYGDYGSNYLYDN

SEQ ID NO: 28 - protein, 15 aa, Artificial sequence
  synthetic
HHSHREFPFYGDYGS

SEQ ID NO: 29 - protein, 13 aa, Artificial sequence
  synthetic
SHREFPFYGDYGS

SEQ ID NO: 30 - protein, 24 aa, Artificial sequence
  synthetic
DSHAKRHHGYKRKFHEKHHSHRGY

SEQ ID NO: 31 - protein, 14 aa, Artificial sequence
  synthetic
GYKRKFHEKHHSHR

SEQ ID NO: 32 - protein, 11 aa, Artificial sequence
  synthetic
SHREFPFYGDY

SEQ ID NO: 33 - protein, 44 aa, Artificial sequence
  synthetic
GGDSHEKRHHGYRRKFHEKHHSHREFPFYGDYGSNYLYDNLPET